(19) United States
(12) Patent Application Publication
(10) Pub. No.: US 2014/0236226 A1
(43) Pub. Date: Aug. 21, 2014

(54) TAILORED POLYMERS

(71) Applicants: University of Bradford, Bradford, Yorkshire (GB); Smith & Nephew PLC, London (GB)

(72) Inventors: Malcolm Brown, Otley (GB); Horacio Montes de Oca Balderas, York (GB); Michael Andrew Hall, York (GB); Phillip Caton-Rose, Bradford (GB); Phillip David Coates, Bradford (GB); Glen Thompson, Bradford (GB)

(73) Assignees: Smith & Nephew PLC, London (GB); University of Bradford, Bradford, Yorkshire (GB)

(21) Appl. No.: 14/350,042

(22) PCT Filed: Oct. 5, 2012
(86) PCT No.: PCT/GB2012/052480

§ 371 Date: Apr. 4, 2014

(30) Foreign Application Priority Data
Oct. 5, 2011 (GB) 1117214.5
Oct. 5, 2011 (GB) 1117216.0
Oct. 5, 2011 (GB) 1117217.8
Oct. 5, 2011 (GB) 1117218.6
Oct. 5, 2011 (GB) 1117219.4
Oct. 5, 2011 (GB) 1117220.2
Oct. 5, 2011 (GB) 1117222.8
Oct. 5, 2011 (GB) 1117223.6
Oct. 5, 2011 (GB) 1117224.4
Nov. 11, 2011 (GB) 1119511.2
May 29, 2012 (GB) 1209510.5

(51) Int. Cl. A61L 24/04; A61L 24/00; A61B 17/00

(52) U.S. Cl. CPC A61L 24/046; A61B 17/00491; A61L 24/0021; A61B 2017/00495
USPC 606/214; 606/1; 514/424; 514/723; 523/118

(57) ABSTRACT

The present invention relates to shape memory polymers (SMP) and especially to shape memory polymer containing a water soluble non-reactive plasticiser and to uses thereof.

### FIELD OF THE INVENTION

The present invention relates to shape memory polymers (SMP) and especially to shape memory polymers containing a water soluble non-reactive plasticiser and to uses thereof

### BACKGROUND OF THE INVENTION

Shape memory polymers (SMPs) have been reported to have various applications in medical devices including stents, fracture fixation devices and fasteners.

Generally, polymers that display shape memory qualities show a large change in modulus of elasticity at the temperatures at which they change shape. The shape-memory function can be achieved by taking advantage of this characteristic.

A mixture of polymer and non-reactive plasticiser is a process known to one skilled in the art, to make a macroscopic body of polymer material. The polymer material that has shape memory qualities which have been programmed into the material can be generated via a process that involves stretching, shearing and/or compressing the material. These processes include, without limitation, zone drawing, hydrostatic extrusion, die drawing, compressive flow moulding, thermoforming, rolling, roll drawing, injection moulding and scorim (Shear Controlled Orientation in Injection Moulding). During this process a defined shape (original shape) is imparted to the macroscopic body. The body may then be softened and deformed into a second shape. The polymer material is then cooled to a temperature where it can retain this second shape. When the polymer material is heated again to a suitable temperature, the shape memory polymer will deform and will try to recover the original shape. Depending on the surrounding media when the SMP recovers its original shape, it can adapt to the surrounding topography.

SMPs are known to activate, changing shape when energy is supplied to these materials or when the activation temperature of the polymer is the same or lower than the temperature of its environment.

Activating an SMP by applying energy is known to result in the material becoming significantly hotter than the surroundings and in certain circumstances this can have a detrimental effect on its surroundings, i.e. biological tissue.

There are numerous references in the prior art which focus on controlling the activation temperature of the SMP by lowering the activation temperature. This has been achieved by either (I) controlling the chemical composition of the polymer chains or by (II) adding a material to lower the activation temperature of the polymer.

Lowering the activation temperature of a polymer is known to have a detrimental effect on important mechanical properties of the material such as mechanical strength, stiffness and long term creep resistance, which will limit the usefulness of the material in certain applications.

To overcome this effect, Gale et al (US 2010/0137471) has reported the use of fast leaching water soluble plasticisers in conventional polymer systems. These plasticisers were reported to leach out at a very high rate such that the polymer does not undergo any detrimental shape changes.

The use of plasticisers in shape memory polymers has also been reported in WO 2008/130916, where a SMP containing long chain fatty acids are responsible for activating the shape memory effect at 37° C. on immersing in water. However, the plasticiser was also reported to accelerate the degradation of the polymer.

It is thus difficult to tailor the thermo-mechanical properties of shape memory polymers since their mechanical properties are correlated with the activation temperature of the SMP. Furthermore, degradable SMP materials need to have certain strength retention profiles during degradation for them to be useful in some applications. Thus, the levels of plasticiser required to generate SMP with activation temperature close to human body temperature may result in the material having inadequate mechanical properties and strength retention profiles during degradation, compromising their use for the manufacture of medical devices, in particular devices for soft tissue fixation for ligament and tendon repair, re-attachment and reconstruction.

There is a need in the art for SMP with low activation temperature characteristics, while maintaining good mechanical properties for soft tissue fixation, particularly bioresorbable and biocompatible shape memory polymers with adequate strength retention during degradation for soft tissue fixation.

### SUMMARY OF THE INVENTION

In one aspect, the present invention provides a polymer composition that is a shape memory polymer composition comprising a polymer and a water soluble plasticiser wherein (i) the plasticiser and polymer are miscible; and (ii) whereupon placing the polymer composition in an aqueous medium results in the polymer softening and deforming where after it hardens.

The plasticiser leaches out from the polymer composition when it is placed in aqueous media, resulting in the polymer composition becoming harder or stiffer. Thus, the invention further provides a shape memory polymer composition comprising a polymer and a water soluble plasticiser, wherein the plasticiser and polymer are miscible. The polymer may be an orientated polymer such that when the polymer composition, i.e. polymer and a water soluble non-reactive plasticiser, is immersed in water the composition softens and deforms, the resulting material then becomes stiffer as the plasticiser diffuses from the polymer composition into the surrounding fluid.

In another aspect, the invention provides a shape memory polymer composition containing a polymer and a water soluble non-reactive plasticiser, wherein the plasticiser and polymer are miscible. The polymer composition can provide shape memory qualities wherein the non-reactive plasticiser leaches out from the polymer composition when it is placed in aqueous media, resulting in the polymer composition becoming stiffer.

The polymer composition as hereinbefore described may comprise an amorphous or semi crystalline material.

In another aspect, a shape memory polymer composition as hereinbefore described wherein the non-reactive plasticiser leaches out from the polymer composition when it is placed in aqueous media, resulting in the polymer composition with adequate thermo-mechanical properties for soft tissue fixation and/or bone repair.

In yet another aspect, the invention a shape memory polymer composition containing a polymer and a water soluble, optionally non-reactive, plasticiser, wherein the plasticiser and polymer are miscible. The shape memory polymer composition can provide shape memory qualities, wherein the, optionally non-reactive, plasticiser leaches out from the shape memory polymer composition when it is placed in living tissue, e.g. a living body, at 37° C., resulting in the shape memory polymer composition having adequate thermo-mechanical properties for soft tissue fixation and/or bone repair.

In another embodiment the, the water soluble plasticiser may comprise a combination of plasticisers having different water solubilities and/or bioactivities.

In another embodiment, the shape memory polymer composition comprising a polymer and a water soluble non-reactive plasticiser is activated by placing it an aqueous environment.

In another embodiment, the shape memory polymer composition comprising a polymer and a water soluble non-reactive plasticiser is activated by supplying energy to the composition.

In another embodiment, the shape memory polymer composition comprising a polymer and a water soluble non-reactive plasticiser comprises a non-degradable polymer.

In another embodiment, the shape memory polymer composition comprising a polymer and a water soluble non-reactive plasticiser comprises a degradable polymer.

In another embodiment the polymer comprises a polylactide and/or polyglycolide based polymers and co-polymers thereof

In another embodiment the shape memory polymer composition contains one or more fillers.

Definitions

By the term “non-reactive plasticiser” is meant a plasticiser which does not significantly accelerate the degradation of the base polymer such that the material has adequate strength retention suitable for soft tissue fixation and bone repair.

Initial glass transition temperature of polymer=Tg0
Glass transition temperature of polymer composition (polymer+plasticiser)=Tg1
Glass transition temperature of polymer composition immersed in water=Tg2
Glass transition temperature of polymer composition immersed in water for 1 wk=Tg3
Initial modulus of elasticity of polymer=E0
Modulus of elasticity of polymer composition (polymer+plasticiser)=E1
Modulus of elasticity of polymer composition immersed in water=E2
Modulus of elasticity of polymer composition immersed in water for 1 week=E3
Unplasticised polymer with Tg0 would require an amount of energy to change shape=K0
Plasticised polymer with Tg1 would require an amount of energy to change shape=K1
Plasticised polymer with Tg2 would require an amount of energy to change shape=K2

### Wherein:

Tg0>Tg1>Tg2

Tg3>Tg2

### And

E0>E1>E2

E3>E2

### And

K0>K1>K2

Adequate strength retention properties during degradation suitable for ligament and tendon repair, re-attachment and reconstruction in the context of the present disclosure means a strength retention profile during degradation similar to a material with clinical history of use.

### DETAILED DESCRIPTION OF THE INVENTION

The following description of the preferred embodiment(s) is merely exemplary in nature and is in no way intended to limit the disclosure, its application, or its uses.

In one embodiment, the invention disclosure relates to a shape memory polymer (SMP) composition comprising a polymer and a water soluble non-reactive plasticiser that enables the polymer to change shape when immersed in aqueous fluid and wherein after shape change the plasticiser diffuses from the shape memory polymer composition into the surrounding fluid and the resulting polymer hardens.

In another embodiment, the invention disclosure relates to a shape memory polymer (SMP) composition comprising a polymer and a water soluble non-reactive plasticiser that enables the polymer to change shape when immersed in aqueous fluid and wherein after shape change the plasticiser diffuses from the shape memory polymer composition into the surrounding fluid and the resulting polymer has adequate mechanical properties for soft tissue fixation and/or bone repair.

In one embodiment, the invention provides a shape memory polymer (SMP) composition comprising a polymer and a water soluble non-reactive plasticiser that enables the polymer to change shape when immersed in aqueous fluid at 37° C. and wherein after shape change the plasticiser diffuses from the shape memory polymer composition into the surrounding fluid and the resulting shape memory polymer hardens.

In another embodiment, the invention provides a shape memory polymer (SMP) composition comprising a polymer and a water soluble non-reactive plasticiser that enables the polymer to change shape when immersed in aqueous fluid at 37° C. and wherein after shape change the plasticiser diffuses from the polymer into the surrounding fluid and the resulting shape memory polymer has adequate mechanical properties for ligament and tendon repair, re-attachment and reconstruction.

In one embodiment, the invention provides a shape memory polymer (SMP) composition comprising a polymer and a water soluble non-reactive plasticiser that enables the polymer to change shape when immersed in a living organism and wherein after shape change the plasticiser diffuses from the shape memory polymer composition into the surrounding fluid and the resulting shape memory polymer hardens.

In another embodiment, the invention provides a shape memory polymer (SMP) composition comprising a polymer and a water soluble non-reactive plasticiser that enables the polymer to change shape when immersed in a living organism and wherein after shape change the plasticiser diffuses from the shape memory polymer composition into the surrounding fluid and the resulting shape memory polymer has adequate mechanical properties for ligament and tendon repair, re-attachment and reconstruction.

In another embodiment, the invention provides a shape memory polymer (SMP) composition comprising a polymer and a water soluble non-reactive plasticiser that enables the polymer to change shape at a lower temperature when activated by the input of energy, wherein after the polymer changes shape and reaches thermal equilibrium with the surrounding media it hardens and wherein the polymer hardens further after the water soluble plasticiser diffuses from the shape memory polymer composition into the surrounding media.

In another embodiment, the invention provides a shape memory polymer (SMP) composition comprising a polymer and a water soluble non-reactive plasticiser that enables the polymer to change shape at a lower temperature when activated by the input of energy, wherein after the polymer changes shape and reaches thermal equilibrium with the surrounding media it hardens and wherein the resulting polymer has adequate mechanical properties for ligament and tendon repair, re-attachment and reconstruction after the water soluble plasticiser diffuses from the shape memory polymer composition into the surrounding media.

In another embodiment, the invention provides a shape memory polymer (SMP) containing composition comprising a polymer and a water soluble non-reactive plasticiser that enables the polymer to change shape when the SMP is placed in an aqueous media at between 37° C. and 80° C., where it hardens as it drops below its activation temperature and wherein the polymer hardens further after the water soluble plasticiser diffuses from the shape memory polymer composition into the surrounding media.

In another embodiment, the invention provides a shape memory polymer (SMP) containing composition comprising a polymer and a water soluble non-reactive plasticiser that enables the polymer to change shape when the SMP composition is placed in an aqueous media at between 37° C. and 80° C., where it hardens as it drops below its activation temperature and wherein the polymer has adequate mechanical properties soft tissue and bone after the water soluble plasticiser diffuses from the shape memory polymer composition into the surrounding media.

In another embodiment, the invention provides a shape memory polymer (SMP) composition comprising a polymer and a water soluble non-reactive plasticiser that enables the polymer to change shape when the SMP composition is placed in a living organism and heated to between 37° C. and 80° C., wherein after the polymer reaches thermal equilibrium with the surrounding media it hardens and wherein the polymer hardens further after the water soluble plasticiser diffuses from the shape memory polymer composition into the surrounding media.

In another embodiment, the invention provides a shape memory polymer (SMP) composition comprising a polymer and a water soluble non-reactive plasticiser that enables the polymer to change shape when the SMP composition is placed in a living organism and heated between 37° C. and 80° C., wherein after the polymer reaches thermal equilibrium with the surrounding media it hardens and wherein the shape memory polymer has adequate mechanical properties for ligament and tendon repair, re-attachment and reconstruction after the water soluble plasticiser diffuses from the shape memory polymer composition into the surrounding media.

In another embodiment, the SMP containing a plasticiser is degradable.

In another embodiment, the SMP containing a plasticiser is non-degradable.

For the purpose of this disclosure the preferred non-degradable polymer is an alkyl substituted polyacrylate, e.g. C1 to 20 alkyl, or a polyurethane and co-polymers or polymer blends thereof. However any biocompatible, non-resorbable, polymeric material may be used, including without limitation, poly(butyl methacrylate), polyethylene terephthalate, poly(ether ether ketone), poly(methyl methacrylate), poly(butylene terephthalate), polyvinyl acetate, polyvinyl alcohol, polyether-co-vinyl alcohols, poly(ester-urethanes), poly(ether-urethane), poly(carbonate-urethane), poly(hydroxy ethyl methacrylate) and copolymers or polymer blends thereof

For the purpose of this disclosure, the preferred resorbable polymer comprises a lactic acid based polymer, e.g. a polylactide based polymer. However any biocompatible, resorbable, polymeric material may be used, including without limitation, poly-alpha-hydroxy acids, polycaprolactones, polydioxanones, polyesters, polyglycolic acid, polylactides, polyorthoester, polyacetals, polyphosphates, polytyrosine carbonates, polyurethanes, polyhydroxy butyrates, polyalkylene succinates, polyanhydrides, polyesteramides and co-polymers or polymer blends thereof.

According to another aspect of the invention there is provided a method of making a shape memory polymer material, said method comprising the steps of:
(i) placing a shape memory polymer composition comprising a polymer and a polymer miscible water soluble plasticiser, in an aqueous environment;
(ii) allowing the water soluble plasticiser to diffuse from the shape memory polymer composition to form a shape memory polymer material;
(iii) allowing shape memory polymer material to soften, deforms and harden.

According to this aspect of the invention placing the shape memory polymer composition in an aqueous environment may comprise placing the shape memory polymer composition in water or an aqueous tissue environment.

According to a yet further aspect of the invention there is provided a method of making a shape memory polymer material, said method comprising the steps of:
(i) forming a shape memory polymer composition by mixing a polymer and a polymer miscible water soluble plasticiser;
(ii) placing the shape memory polymer composition, in an aqueous environment;
(iii) allowing the water soluble plasticiser to diffuse from the shape memory polymer composition to form a shape memory polymer material;
(iv) allowing shape memory polymer material to soften, deforms and harden.

In another embodiment, the invention disclosure relates to a material comprising a shape memory polymer composition, the composition comprising a lactic acid based polymer (PLA) and a water soluble non-reactive plasticiser, wherein the plasticiser and polymer are miscible and the resulting polymer composition provides shape memory qualities and the plasticiser leaches from the shape memory polymer composition after activation resulting in the material hardening.

In another embodiment the water soluble non-reactive plasticiser comprises from about 0.1 to 30% by weight of the shape memory polymer composition.

In another embodiment the water soluble non-reactive plasticiser comprises between about 0.5-10% by weight of the shape memory polymer composition.

In another embodiment the water soluble non-reactive plasticiser comprises between about 2.0-6% by weight of the shape memory polymer composition.

In another embodiment the water soluble non-reactive plasticiser is biologically active.

In another embodiment the plasticiser is osteoconductive, osteoinductive, angiogenic or antimicrobial.

In an another embodiment the water soluble non-reactive plasticiser is a material that, when mixed in a 5% weight/weight mixture poly(L/DL) lactide (70/30, IV 3.8), results in a material with (i) a strength retention greater than 10 wk; and (ii) an increase in modulus after degrading the sample for 1 week in vitro (37° C./water).

In another embodiment the water soluble non-reactive plasticiser comprises one or more of the following: esters of citric acid e.g. propylene glycol, polypropylene glycol, dipropylene glycol, tripropylene glycol, triethyl citrate, N-methyl pyrrolidone (NMP), dimethyl formaldehyde (DMF), dimethyl sulphoxide (DMSO), glyceryl triacetate, ethyl acetate, ethyl lactate, ethyl glyconate, ethyl adipate, ethyl 6-hydoxy adipate, 1,3-dimethyl-2-imidazolodinone (DMI), N,N-dimethylacetamide (DMF), 1,3-dimethyltetrahydropyrimodinone (DMPU), oligomers of ethylene glycol, polyethylene glycol, polypropylene oxide, esters of hyaluronic acid, glycerol, esters of glycerol, polyvinyl alcohol, poly(vinyl pyrrolidone).

In another embodiment the polymeric material may comprise a composite or matrix having reinforcing material and/or phases in the form of fibres, meshes, particulates, rods and/or platelets. For the purpose of this disclosure, when a reinforcing material is present, the reinforcing material may comprise a polymeric material, ceramic, glass or metallic material, or a mixture of these. Furthermore, the reinforcing material may include, inorganic fibres, polymeric fibres, metallic fibres, inorganic particulates, polymeric particulates, metallic particulates and these may be, without limitation, calcium sulphate, magnesium sulphate, bioceramics, calcium phosphate, hydroxy apatite, calcium carbonate, tricalcium phosphate, degradable magnesium metal, calcium phosphate glasses, degradable silicon based glasses, polylactide fibres, polylactide particulates, polyglycolic acid fibres and mixtures thereof. Other reinforcing material or phases known to one ordinary skilled in the art may also be used.

In another embodiment the polymeric material may be porous or contain a porogen filler. These porogens may be in the form of particles and/or fibres and include, without limitation, water soluble polymers, inorganic solids and organic salts such as sodium chloride, calcium sulphate, calcium lactate, magnesium lactate, amorphous calcium carbonate, calcium glyconate, magnesium glyconate, salts of amino acids, sodium glutonate, sodium hydrogen carbonate, sucrose, polyvinylalcohol, polysaccharides, hyaluronic acid, polyalkylene oxides, polyethylene glycol and blends thereof. The filler may comprise an osteoconductive, osteoinductive, angiogenic or antimicrobial material.

In another embodiment, one or more active agents may be incorporated into the material. Suitable active agents include, but shall not be limited to, bone morphogenic proteins, antibodies, anti-inflammatory agents, angiogenic factors, monobutyrin, thrombin, modified proteins, platelet rich plasma, bone marrow aspirate and any cell source from flora or fauna, such as living cells, preserved cells, dormant cells and dead cells.

In another embodiment the polymer material may include a polylactide acid based polymer containing lactic acid, L-lactide (PLLA), D-lactide, D,L-lactide, glycolide, glycolic acid, caprolactone, capronic acid, dioxanone and/or trimethylene carbonate monomers.

In another embodiment the polymer is a poly lactic acid containing L- and D,L-lactide.

In another embodiment the polymer is polylactic acid containing L- and D,L-lactide containing 60-80% L- and 20-40% D,L-Lactide.

In another embodiment, the shape memory polymer composition comprises a polymer and a water soluble non-reactive plasticiser which may be used to make devices, such as, without limitation, implantable devices, e.g. rods, pins, screws, plates, nails, anchors, wedges and meshes for use in bone and/or soft tissue repair.

Thus, the invention further provides a medical device, such as an implantable device, comprising a shape memory polymer composition including a polymer and a water soluble non-reactive plasticiser wherein the device is activated on placing in water, followed by the leaching of the plasticiser resulting in the device becoming stiffer/harder The implantable device according to this aspect of the invention may comprise one or more of a rod, pin, screw, plate, nail, anchor, wedge or mesh, for example, for use in bone and/or soft tissue repair.

The invention further provides a method of producing a medical article comprising a shape memory polymer composition, said shape memory polymer composition comprising a polymer and a water soluble plasticiser, wherein the plasticiser and polymer are miscible, wherein the method comprises combining a polymer with a water soluble plasticiser.

The invention further provides a method for fixating soft tissue to bone which involves creating a cavity in bone, placing a device containing a shape memory polymer composition or an SMP as hereinbefore described, and at least one cable where upon aqueous media diffuses into the device causing the material to change shape and then harden.

The invention further provides a method for fixating soft tissue to bone which involves creating a cavity in bone, placing a device containing a polymer composition containing a miscible water soluble plasticiser (or an SMP as hereinbefore described), and at least one cable where the device is heated resulting in a shape change, allowed to cool whereupon the material harden followed by aqueous media diffusing into the device causing the harden further.

The invention further provides the use of a shape memory polymer composition or an SMP as hereinbefore described in the manufacture of a fixation device for use in bone and/or soft tissue repair.

The shape memory polymer composition of the invention desirably contains a plasticiser that self-deforms in the presence of liquids, wherein after deformation the polymer hardens at the same temperature of the environment it deformed.

In another embodiment, the shape memory polymer composition comprising a polymer and a water soluble non-reactive plasticiser may be shaped into a sleeve, plug or other appropriate geometry, such that these materials can be combined with, or incorporated into, a fixation device otherwise lacking shape memory qualities.

In another embodiment, the shape memory polymer composition of the invention may be used in combination with other SMP or non-SMP materials to make medical devices such as, medical implants, for example, without limitation, rods, pins, screws, plates nails, anchors, wedges, and meshes for use in bone and soft tissue repair.

In another embodiment, the energy supplied to the shape memory polymer composition of the invention to activate the shape change includes electrical and thermal sources, the use of force, or mechanical energy, electromagnetic and/or a solvent. The thermal energy may include a heated liquid, such as water or saline. It is also within the scope of the disclosure that once the shape memory material is placed in a living organism, heat would be transferred from blood, tissue and in general from the surrounding media to provide the energy necessary to cause shape change of the shape memory material.

In another embodiment the plasticiser of choice may be comprised a combination of plasticisers having different water solubilities and/or bioactivities.

In another embodiment, the water soluble non-reactive plasticiser is a material that forms a single phase with the polymer such that the resulting material can be orientated, resulting in the lowering of the glass transition temperature (Tg) of the base polymer such that the material changes shape when placed in aqueous fluid at 37° C., without substantially having a detrimental reduction in the strength retention during degradation of the polymer and wherein the resulting material becomes stiffer when the plasticiser diffuses out from the polymer.

In another embodiment of the invention, the plasticiser lowers the glass transition temperature of the SMP from Tg0 to Tg1, which in turn decreases even further on immersion in aqueous media to Tg2, causing shape change of the SMP. However Tg2 increases thereafter to Tg3 as the plasticiser diffuses from the polymer.

In another embodiment the activation temperature of the polymer composition is reduced by the addition of the water soluble non-reactive plasticiser by 1° C. to 100° C. depending upon, inter alia, the polymer, plasticiser type and loading level.

In another embodiment, the water soluble non-reactive plasticiser reduces the glass transition temperature of the polymer. High concentrations of the plasticiser will reduce the transition temperature and mechanical properties to the point where the shape memory properties are compromised. For the purpose of this disclosure, a high concentration of non-reactive plasticiser would be represented by more than 30% by weight of the polymer composition. Therefore the non-reactive plasticiser is preferred to be between 0.5% and 10% by weight of the shape memory polymer composition and, in some circumstances, is between 2% and 6% by weight of the composition. The ratio of non-reactive plasticiser is dependent upon the polymer and non-reactive plasticiser used.

In another embodiment, the shape memory polymer composition has adequate strength retention properties during degradation soft tissue fixation and bone repair.

In another embodiment, the invention provides a shape memory polymer (SMP) composition comprising a polymer and a water soluble non-reactive plasticiser that enables the polymer to change shape in less than 1 week when immersed in aqueous fluid and wherein after shape change the plasticiser diffuses from the polymer into the surrounding fluid and the resulting polymer hardens.

In another embodiment, the invention disclosure relates to a shape memory polymer (SMP) containing a water soluble non-reactive plasticiser that enables the polymer to change shape in less than 1 week when immersed in aqueous fluid and wherein after shape change the plasticiser diffuses from the polymer into the surrounding fluid and the resulting polymer has adequate mechanical properties for soft tissue fixation and bone repair.

In one embodiment, the invention disclosure relates to a shape memory polymer (SMP) containing a water soluble non-reactive plasticiser that enables the polymer to recover 70% or more of its original shape within 1 week when immersed in aqueous fluid at 37° C. and the resulting polymer has adequate mechanical properties for soft tissue fixation and bone repair.

In one embodiment, the invention disclosure relates to a shape memory polymer (SMP) composition containing a water soluble non-reactive plasticiser that enables the polymer to recover 70% or more of its original shape in less than 24 hours when immersed in aqueous fluid at 37° C. and the resulting polymer has adequate mechanical properties for soft tissue fixation and/or repair.

In one embodiment, the invention disclosure relates to a shape memory polymer (SMP) composition containing a water soluble non-reactive plasticiser that enables the polymer to recover 90% or more of its original shape in less than 24 hours when immersed in aqueous fluid at 37° C. and the resulting polymer has adequate mechanical properties for soft tissue fixation and/or repair.

In another embodiment, the invention disclosure relates to a shape memory polymer (SMP) composition containing a water soluble non-reactive plasticiser that enables the polymer to change shape in less than 1 week when immersed in a living organism and wherein, after shape change, the plasticiser diffuses from the shape memory polymer composition into the surrounding fluid and the resulting polymer has adequate mechanical properties for soft tissue fixation and/or bone repair.

### BRIEF DESCRIPTION OF THE DRAWINGS

FIG. 1 shows a schematic diagram of the description of one of the embodiments of the invention;

FIG. 2 shows a schematic diagram of the description of one of the embodiments of the invention;

FIG. 3 shows a schematic diagram of the thermo-mechanical properties of the polymer;

FIG. 4 shows a graph of the mechanical properties of the SMP with tripropylene glycol (TPG) plasticiser measured at 37° C. using an Instron™ 6684 tensile testing machine fitted with SFL oven (45° C.) after 0, 1 and 3 days; and

FIG. 5 shows a graph of the mechanical properties of the SMP with NMP plasticiser measured at 37° C. using an Instron™ 6684 tensile testing machine fitted with SFL oven (45° C.) after 0, 1 and 3 days.

### DETAILED DESCRIPTION OF THE DRAWINGS

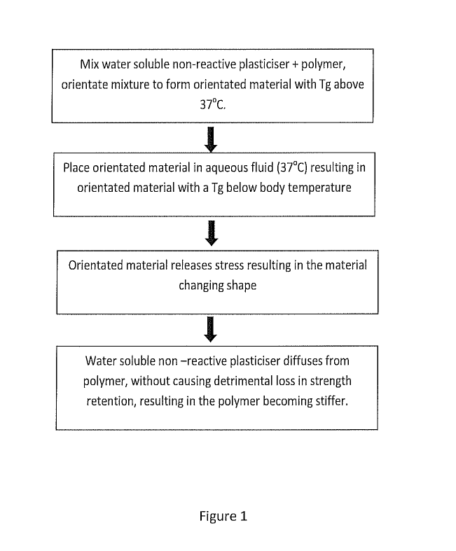

FIG. 1 describes the general process and properties of our SMP materials that are design to activate at body temperature

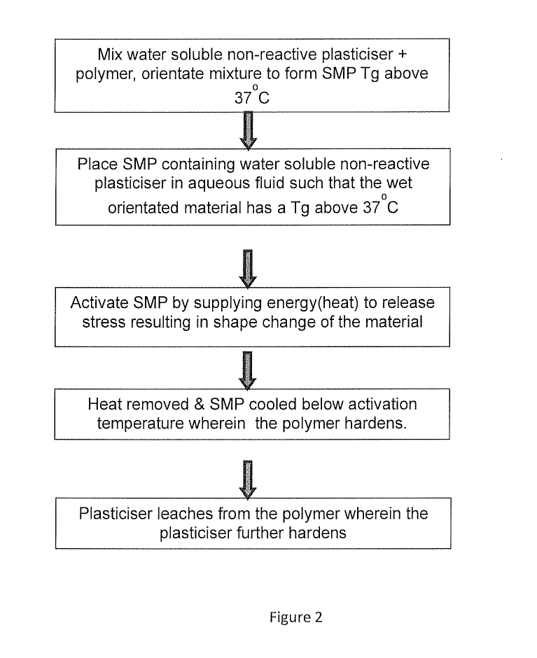

FIG. 2 describes the general process and properties of our SMP material that are designed to activate above body temperature

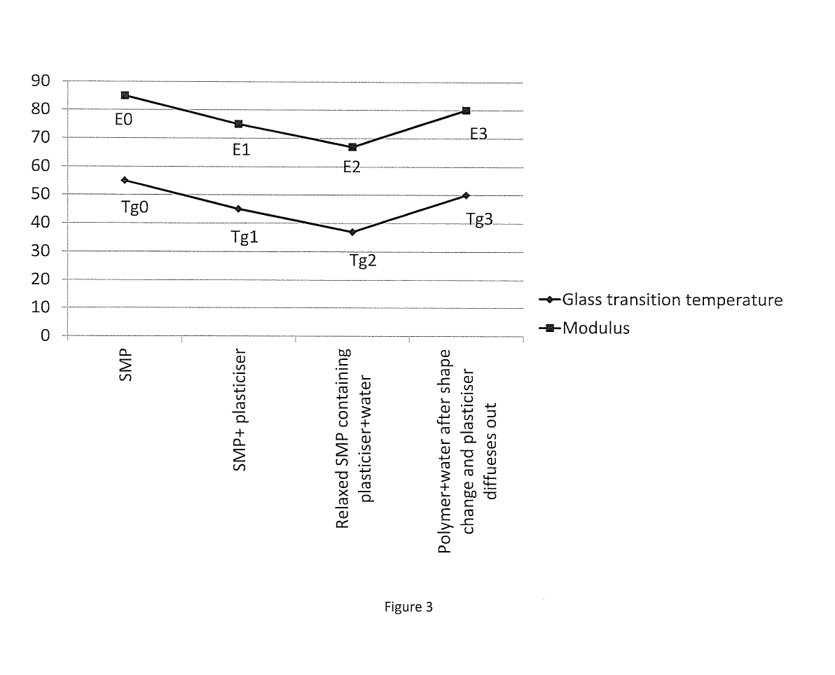

FIG. 3 describes the effect of plasticiser on the glass transition temperature, modulus of elasticity and energy required to activate the SMP. The glass transition temperature of the SMP without plasticiser decreases from Tg0 to Tg1 when the plasticiser is added. When immersed into aqueous media the glass transition temperature of the SMP decreases further to Tg2. However, when SMP changes shape and plasticiser diffuses out of the polymer, the glass transition temperature of the polymer increases to Tg3, which is greater than Tg2.

The modulus of elasticity of the SMP without plasticiser decreases from E0 to E1 when the plasticiser is added. When immersed into aqueous media the modulus of the SMP decreases further to E2. However, when SMP changes shape and the plasticiser diffuses out of the polymer, the modulus of the polymer increases to E3, which is greater than E2.

### Examples

### Experiment 1

### SMP Materials Containing Tripropylene Glycol Plasticiser

(I) Sample preparation

Polylactide (Purac™ 7038, 100 g) was dried in a Motan™ for 4 hrs at 50° C. The polymer was cooled and placed into a 500 ml jar. Tripropylene glycol (TPG) (11 g, 5.5 g %) was added to the polymer, via the use of a syringe, and the mixture manually agitated. The jar was sealed and the polymer/plasticiser mixture was then further mixed for 30 mins, using an orbital shaker. The material was melt blended using a prim twin screw extruder (100 rpm/150° C.) to yield polymer pellets. Dumbbells were generated by compression moulding 30 g of the this material using a hot press (170° C., 20 tonnes) to form a polymer sheet from which dumbbells samples were stamped out.

(II) SMP generation

SMP samples were generated by placing a PLA/TPG dumbbell described in 1(I) into an Instron™ 6684 tensile testing machine fitted with SFL oven (45° C.) and stretched (drawing speed of 300 mm/min) to yield a material with a draw ratio of 5:1.

(III) SMP Relaxation properties

PLLA/TPG SMP test samples generated in 1(II) were placed in glass vials and filled with water. The vials were placed in a water bath (37° C.) and allowed to equilibrate; recovery, tensile properties and TPG content of the samples were evaluated over a period of days.

The polylactide/TPG samples were found to relax after 2 hr immersion in water with a recovery of over 90%.

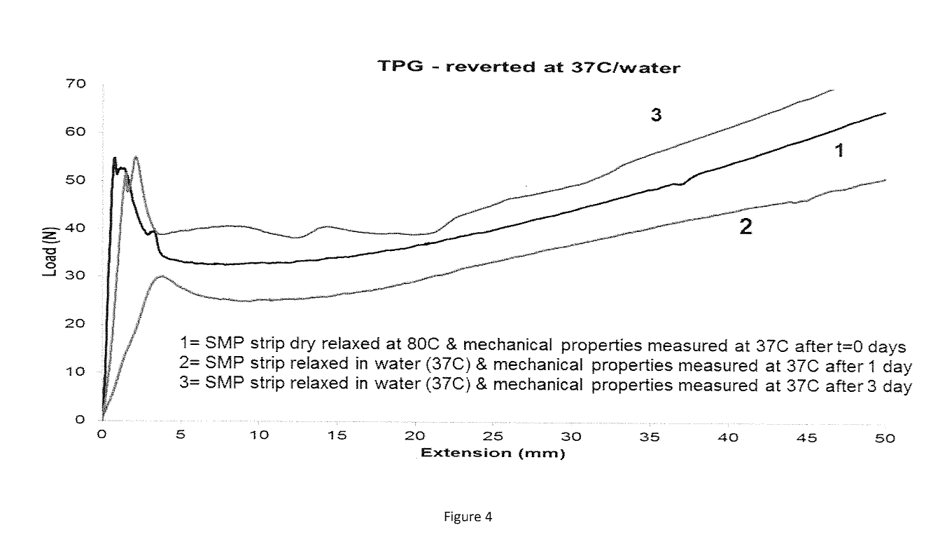

Mechanical properties of the SMP were measured at 37° C. using an Instron™ 6684 tensile testing machine fitted with SFL oven (45° C.).This data is shown in FIG. 4.

### Experiment 2

### SMP Materials Containing NMP Plasticiser

(II) Sample preparation

Polylactide (Purac™ 7038, 200 g) was dried in a Motan™ for 4 hrs at 50° C. The polymer was cooled and placed into a jar containing 500 ml jar. Anhydrous N-methyl pyrrolidone(NMP) (10 g) was added to the polymer, via the use of a syringe, and the mixture manually agitated. The jar was sealed and the polymer/plasticiser mixture was then further mixed for 30 mins using an orbital shaker. The material was melt blended using a prim twin screw extruder (100 rpm/150° C.) to yield polymer pellets. Dumbbells were generated by compression moulding 30 g of the this material using a hot press (170° C.) to form a polymer sheet from which dumbbells samples were stamped out. NMP content in the polymer samples was determined using gas chromatography (GC). The samples were found to contain 4.5% wt/wt of NMP.

(IV) SMP generation.

SMP samples were generated by placing PLLA/NMP dumbbells described in 2(I) into an Instron™ 6684 tensile testing machine fitted with SFL oven (45° C.) and stretched (300 mm/min) to yield a material with a draw ratio of 5:1.

(V) SMP Relaxation properties

SMP test samples generated in 2(II) were placed in glass vials and filled with water. The vials were placed in a water bath (37° C.) and allowed to equilibrate; Recovery, tensile properties and NMP content of the samples were evaluated over a period of days.

The Polylactide/NMP samples were found to relax after 2 hr immersion in water with a recovery of over 90%.

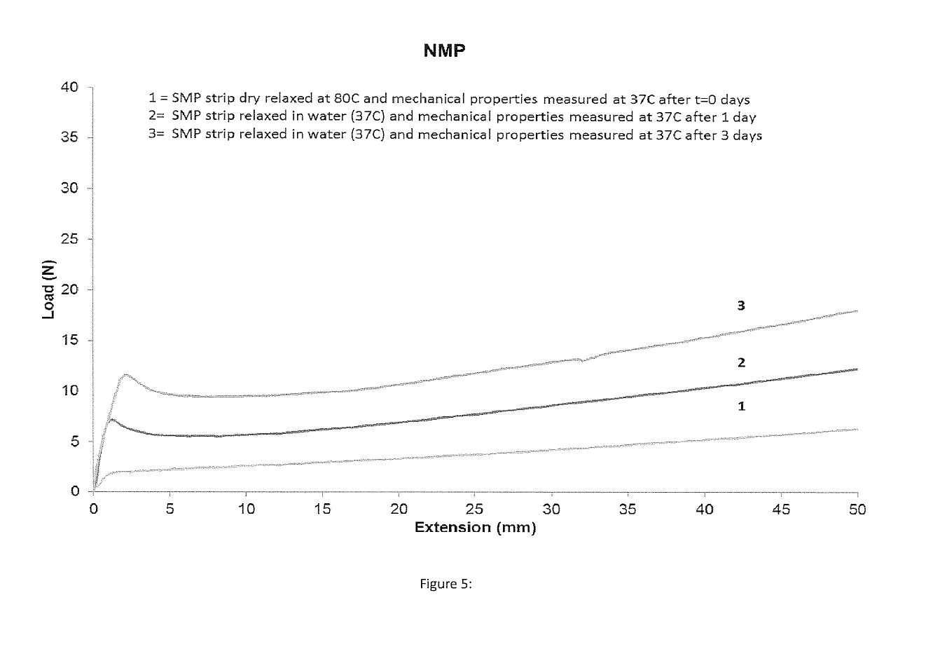

Mechanical properties of the SMP were measured at 37° C. using an Instron™ 6684 tensile testing machine fitted with SFL oven (45° C).This data is shown in FIG. 5.

1-58. (canceled)

59. A polymer composition comprising a polymer and a water soluble plasticizer wherein the plasticizer and polymer are miscible and where on immersion in an aqueous medium the polymer composition softens, deforms, the plasticizer diffuses in the aqueous medium and the resulting material then becomes stiffer.

60. The polymer composition according to claim 59 wherein the plasticizer diffuses from the polymer in a fluid phase.

61. The polymer composition according to claim 59 in which the composition is degradable and/or resorbable.

62. The polymer composition according to claim 59 wherein the polymer comprises a lactic acid based polymer or co-polymers or polymer blends thereof

63. The polymer composition according to claim 59 wherein the polymer comprises a polylactic acid containing L- and D,L-lactide.

64. The polymer composition according to claim 59 wherein the polymer comprises a polylactic acid containing 60-80% L- and 20-40% D,L-Lactide.

65. The polymer composition according to claim 59 in which the composition is non-degradable.

66. The polymer composition according to claim 65 wherein the non-degradable polymer is an alkyl substituted polyacrylate or a polyurethane; and co-polymers or polymer blends thereof

67. The polymer composition according to claim 59 wherein the water soluble plasticizer is a non-reactive plasticizer.

68. The polymer composition according to claim 67 wherein the plasticizer comprises from about 0.1 to 50% by weight of the polymer composition.

69. The polymer composition according to claim 59 in which the water soluble plasticizer forms a single phase with the polymer.

70. The polymer composition according to claim 59 in which the plasticizer comprises a combination of plasticizers having different water solubilities and/or bioactivities.

71. The polymer composition according to claim 59 wherein the composition is capable of being activated by placing it an aqueous environment.

72. The polymer composition according to claim 59 in which the water soluble non-reactive plasticizer has osteoconductive, osteoinductive, angiogenic, and/or antimicrobial properties.

73. The polymer composition according to claim 59 wherein the polymer comprises at least one of polylactide, polyglycolide, and co-polymers thereof

74. The polymer composition according to claim 59 which contains filler that enhances the mechanical properties of the polymer composition.

75. The polymer composition according claim 74 in which the filler has biologic properties including osteoconductive, osteoinductive, angiogenic and/or antimicrobial properties.

76. The polymer composition according to claim 74 in which the filler is in the form of at least one of particulates, fibers, or mesh.

77. A shape memory polymer (SMP) composition comprising at least one SMP and a water soluble non-reactive plasticizer, the plasticizer enabling the SMP to change shape at an activation temperature between about 20° C. to 90° C. when the SMP is placed in an aqueous media, the SMP hardening if its temperature drops below its activation temperature, and the SMP hardening further as the water soluble plasticizer diffuses from the SMP composition.

78. The shape memory polymer composition according to claim 77 in which the composition is degradable.

79. The shape memory polymer composition according to claim 77 in which the composition is non-degradable.

80. The shape memory polymer composition according to claim 90 in which the water soluble non-reactive plasticizer forms a single phase with the polymer.

81. The shape memory polymer composition according to claim 77 in which the water soluble non-reactive plasticizer is biologically active and has osteoconductive, osteoinductive, angiogenic and/or antimicrobial properties.

82. The shape memory polymer composition according to claim 77 which contains a filler that enhances the mechanical properties of the polymer.

83. A method of making a shape memory polymer material comprising:
(i) placing a shape memory polymer composition comprising a polymer and a polymer miscible water soluble plasticizer, in an aqueous environment;
(ii) allowing the water soluble plasticizer to diffuse from the shape memory polymer composition to form a shape memory polymer material;
(iii) allowing shape memory polymer material to soften, deform and harden.

84. A medical device comprising a polymer composition according to claim 59.

85. The medical device according to claim 59 wherein the medical device is for use as a fixation device in bone and soft tissue repair.

86. A method for fixating soft tissue to bone which involves creating a cavity in bone, placing a device containing a polymer composition according to claim 77, and at least one cable where upon aqueous media diffuses into the device causing the material to change shape and then harden.

87. The polymer composition according to claim 59 wherein the plasticizer is tripropylene glycol and/or NMP.
